(19) United States
(12) Patent Application Publication
(10) Pub. No.: US 2010/0055154 A1
(43) Pub. Date: Mar. 4, 2010

(54) COAXIAL ELECTROSPUN FIBERS AND STRUCTURES AND METHODS OF FORMING THE SAME

(76) Inventors: I-Chien Liao, Durham, NC (US); Kam W. Leong, Durham, NC (US); Sing-Yian Chew, Singapore (SG)

Correspondence Address: MYERS BIGEL SIBLEY & SAJOVEC, PO BOX 37428, RALEIGH, NC, 27627, US

(21) Appl. No.: 12/373,737

(22) PCT Filed: Jul. 18, 2007
(86) PCT No.: PCT/US07/16263

§ 371 Date: Oct. 6, 2009

Related U.S. Application Data
(60) Provisional application No. 60/832,779, filed on Jul. 24, 2006.

(51) Int. Cl. A61K 9/70; B29C 47/02; A61P 43/00

(52) U.S. Cl. 424/443; 264/465

(57) ABSTRACT

Nanofibers and microfibers having a core and a polymer shell surrounding the core are provided. The shell includes a plurality of channels that extend from an outer shell surface to the core, and one or more agents, such as pharmacological materials, proteins, viruses, plasmid DNA, bacterial cells, drug-loaded nanoparticles, are encapsulated within the core. The one or more agents discharge from the core through the channels at a controlled rate. The channels are formed by porogen material within the polymer shell.

### RELATED APPLICATION

This application claims the benefit of and priority to U.S. Provisional Patent Application No. 60/832,779, filed Jul. 24, 2006, the disclosure of which is incorporated herein by reference as if set forth in its entirety.

### FIELD OF THE INVENTION

The present invention relates generally to fibers and, more particularly, to electrospun fibers.

### BACKGROUND OF THE INVENTION

Intraluminal devices, such as stents, are typically used as adjuncts to percutaneous transluminal balloon angioplasty procedures in the treatment of occluded or partially occluded arteries and other blood vessels. A stent functions as scaffolding to structurally support a vessel wall and thereby maintain luminal patency. It may be desirable to provide localized pharmacological treatment of a vessel at a site being supported by a stent. Thus, sometimes it is desirable to utilize a stent both as a support for a lumen wall as a well as a delivery vehicle for one or more pharmacological agents. Unfortunately, metallic materials typically employed in conventional stents are not generally capable of carrying and releasing pharmacological agents. Previously devised solutions to this dilemma have been to join drug-carrying polymers to metallic stents.

Although viral gene transfer is efficient in achieving transgene expression for tissue engineering applications, drawbacks of virus dissemination, toxicity, acute immune response and transient gene expression have hindered its success. Most tissue engineering studies thus opt to genetically engineer cells in vitro prior to their introduction in vivo. However, it would be attractive to be able to transfect the infiltrating progenitor cells in situ and obviate the need for in vitro manipulation.

The complex process of tissue morphogenesis involves the coordinated delivery of biochemical and topographical cues. Nanofibrous meshes can provide nano-topographical cues that stimulate cells in a manner drastically different from that of films and microscale fibrous scaffolds. To further improve the capability of nanofibers as tissue engineering scaffolds, co-axial electrospinning has been proposed to fabricate drug-encapsulated nanofibrous meshes with enhanced drug loading capacity. However, the degree of control over the release kinetics from these co-axially electrospun fibers has been limited.

### SUMMARY

According to some embodiments of the present invention, a layer of fibrous material includes a plurality of fibers, wherein each fiber comprises a core and a polymer shell surrounding the core. The shell includes a plurality of channels that extend from an outer shell surface to the core, and an agent (e.g., pharmacological materials, proteins, viruses, plasmid DNA, bacterial cells, drug-loaded nanoparticles, etc.), is encapsulated within the core. The agent discharges from the core through the channels at a controlled rate.

According to other embodiments of the present invention, a tissue engineering scaffold is formed from a plurality of fibers, wherein each fiber comprises a core and a polymer shell surrounding the core. The shell of each fiber includes a plurality of channels that extend from an outer shell surface to the core. Viral particles are encapsulated within the core and discharge from the core through the channels at a controlled rate. In some embodiments, the viral particles are substantially uniformly distributed within the core. Cells seeded on the scaffold exhibit transgene expression for a predetermined period of time. Tissue engineering scaffolds embedded with proteins, according to embodiments of the present invention, may synergistically present topographical and biochemical signals to cells for tissue engineering applications.

According to other embodiments of the present invention, a layer of fibrous material includes a plurality of fibers, wherein each fiber comprises a core and a polymer shell surrounding the core. The shell includes a plurality of channels that extend from an outer shell surface to the core. Viable bacterial cells are encapsulated within the core in an aqueous solution. In some embodiments, the bacterial cells secrete material through the channels at a controlled rate. In other embodiments, the bacterial cells absorb material external to the fibers through the channels. In other embodiments, the bacterial cells discharge from the core through the channels at a controlled rate.

In each of the various embodiments, channels in each fiber are formed by porogen material, such as polyethylene glycol (PEG), disposed within the polymer shell. PEG is non-cytotoxic and is easily filtered by the kidney at MW<10,000. In addition, the wide range of molecular weights available for PEG affords an opportunity to finely manipulate the nanoporous structure of fiber shells, thereby controlling the rate of discharge of agents from the fiber cores.

In each of the various embodiments, the shell surrounding the core of each fiber may be poly(caprolactone) (PCL). In each of the various embodiments, the plurality of fibers may be aligned or may be randomly arranged. In each of the various embodiments, the fibers may be nanofibers or microfibers.

According to some embodiments of the present invention, a method of forming a fibrous material includes co-axially electrospinning first and second solutions to form a plurality of fibers. The first solution forms a fiber core and the second solution forms a shell surrounding the core. The first solution includes an agent selected from the group consisting of pharmacological materials, proteins, viruses, plasmid DNA, bacterial cells, and drug-loaded nanoparticles, and the second solution is a polymeric solution that includes porogen material. The porogen material is configured to leach from the shell and form a plurality of channels that extend from an outer shell surface to the core.

### BRIEF DESCRIPTION OF THE DRAWINGS

The accompanying drawings, which form a part of the specification, illustrate key embodiments of the present invention. The drawings and description together serve to fully explain the invention.

FIG. 1A is a block diagram that illustrates an apparatus and method for producing a core-shell fiber via coaxial electrospinning, according to some embodiments of the present invention.

FIG. 1B illustrates a first needle concentrically surrounding a second needle in the apparatus of FIG. 1A.

FIG. 2 is a cross-sectional view of a nanofiber, according to some embodiments of the present invention, having channels that have been created by porogen material leaching from the core thereof.

FIGS. 3A-3J are electron microscopy images of electrospun nanofibers according to some embodiments of the present invention.

FIG. 4A is a graph that illustrates the controlled release of encapsulated bovine serum albumin from PCL and various formulations of PEG blended PCL nanofibers, according to some embodiments of the present invention.

FIG. 4B is a fluorescent microscopy image of aligned FITC-BSA loaded PCL fibers.

FIG. 4C is a corresponding phase image of aligned FITC-BSA loaded PCL fibers.

FIG. 5A is a graph that illustrates the controlled release of encapsulated PDGF-bb from PCL nanofibers and PCL-PEG (7% PCL+20 mg/mL PEG MW 3400)) nanofibers.

FIG. 5B is a graph that illustrates bioactivity of PDGF-bb released into the supernatant solution based on the enhanced proliferation rate of NIH 3T3 cells.

FIGS. 6A-6B are fluorescent images of bovine pulmonary artery smooth muscle cells seeded on nanofibrous scaffolds, according to some embodiments of the present invention.

FIGS. 7A-7D are electron microscopy images of coaxially electrospun fibers according to some embodiments of the present invention.

FIGS. 8A-6H are electron microscopy images of coaxially electrospun fibers according to some embodiments of the present invention.

FIGS. 9A-9D are graphs illustrating performance of virus-encapsulated electrospun fibers, according to some embodiments of the present invention.

FIG. 10A is a bar chart illustrating transgene expression in seeded cells, according to some embodiments of the present invention.

FIGS. 10B-10C are fluorescence microscopy images of fibers according to some embodiments of the present invention.

### DETAILED DESCRIPTION

The present invention now is described more fully hereinafter with reference to the accompanying drawings, in which preferred embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

Like numbers refer to like elements throughout. In the figures, the thickness of certain lines, layers, components, elements or features may be exaggerated for clarity. Broken lines illustrate optional features or operations unless specified otherwise. All publications, patent applications, patents, and other references mentioned herein are incorporated herein by reference in their entireties.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. As used herein, phrases such as “between X and Y” and “between about X and Y” should be interpreted to include X and Y. As used herein, phrases such as “between about X and Y” mean “between about X and about Y.” As used herein, phrases such as “from about X to Y” mean “from about X to about Y.”

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the specification and relevant art and should not be interpreted in an idealized or overly formal sense unless expressly so defined herein. Well-known functions or constructions may not be described in detail for brevity and/or clarity.

It will be understood that when an element is referred to as being “on”, “attached” to, “connected” to, “coupled” with, “contacting”, etc., another element, it can be directly on, attached to, connected to, coupled with or contacting the other element or intervening elements may also be present. In contrast, when an element is referred to as being, for example, “directly on”, “directly attached” to, “directly connected” to, “directly coupled” with or “directly contacting” another element, there are no intervening elements present. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is inverted, elements described as “under” or “beneath” other elements or features would then be oriented “over” the other elements or features. Thus, the exemplary term “under” can encompass both an orientation of “over” and “under”. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

It will be understood that, although the terms “first”, “second”, etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a “first” element, component, region, layer or section discussed below could also be termed a “second” element, component, region, layer or section without departing from the teachings of the present invention. The sequence of operations (or steps) is not limited to the order presented in the claims or figures unless specifically indicated otherwise.

The term “agent” shall include any and all types of materials encapsulated within fibers (e.g., nanofibers, microfibers), according to embodiments of the present invention.

Electrospinning is a technology which utilizes electrical charge to overcome the surface tension of a polymer solution in order to shear the polymer solution into micro-to-nanoscale fibers. Fibers having diameters that are less than one micron are referred to as “nanofibers”. Fibers having diameters equal to or greater than one micron are referred to as microfibers.

Co-axial electrospinning involves encapsulating an aqueous phase solution of material (e.g., drugs, proteins, viruses, bacterial cells, etc.) into the core of electrospun fibers.

According to some embodiments of the present invention, poly(caprolactone), an FDA-approved polymer for in vivo application, can be produced into microfibers and nanofibers (hereinafter collectively referred to as “fibers”) in such fashion. According to some embodiments of the present invention, coaxial electrospinning is used to incorporate drugs of interest into fibers to provide a controlled release over time. Porogens are included in the shell of the electrospun structure to achieve control over the release rate of the drug from the fibers. Fiber structures, according to embodiments of the present invention, can achieve prolonged drug delivery and can be used as tissue engineering scaffolds.

According to embodiments of the present invention, co-axial electrospinning is an efficient method of encapsulating proteins into fibers without compromising bioactivity. PEG introduced into the shell of PCL fibers can serve as a porogen, and the rate of protein release is dependent on the molecular weight and concentration of the PEG. In addition, co-axial electrospinning allows the encapsulation of bioactive agents such as hydrophilic drugs, proteins and growth factors. This is a feature that is not attainable with conventional electrospinning due to the immiscibility of hydrophilic drugs and proteins in organic solvents. Co-axial electrospinning not only can encapsulate the hydrophilic drugs and proteins efficiently, but also preserves their bioactivity and delivers them on cue.

According to some embodiments of the present invention, protein loaded nanofibers can be aligned to provide nano-topographical cues to cells of interest. Such aligned, protein loaded nanofibers can be a significant advancement in scaffold design for specific tissue engineering applications.

According to some embodiments of the present invention, poly(ethylene oxide) is included into the shell of electrospun poly(caprolactone) nanofibers as a method to control the release of drugs and protein encapsulated into the nanofibers. Heretofore, protein encapsulation into nanofibers has not included porogen into the shell of nanofibers. Applicants have unexpectedly discovered that the inclusion of porogen, which results in the formation of pores on the surface of the nanofibers, allows the release of encapsulated nanoscale particles, which is not possible with non-porogen included nanofibers.

Embodiments of the present invention can be used to provide prolonged bioactive signaling release through nanofibers, providing the seeded cells both nanotopographic and biochemical signals to push the cells of interest into their designated tissue lineage.

Embodiments of the present invention can be used as a long term drug release vehicle in vivo.

Co-axial electrospinning, according to some embodiments of the present invention, is a process that can efficiently encapsulate proteins and produce aligned fibers. The inclusion of a porogen (e.g., PEG) in the shell of fibers can provide versatility in the release of a drug (or other material) of interest. The inclusion of PEG as a porogen allows the release of protein of interest, independent of the core diameter or protein type. The co-axial electrospinning technique can also be applied to the encapsulation of particles, viruses, or bacterial cells, where the release will be dependent on the pore formation on the surface of nanofibers. According to other embodiments of the present invention, aligned drug loaded fibers can be used as support scaffolds in applications that require high level of cell orientation.

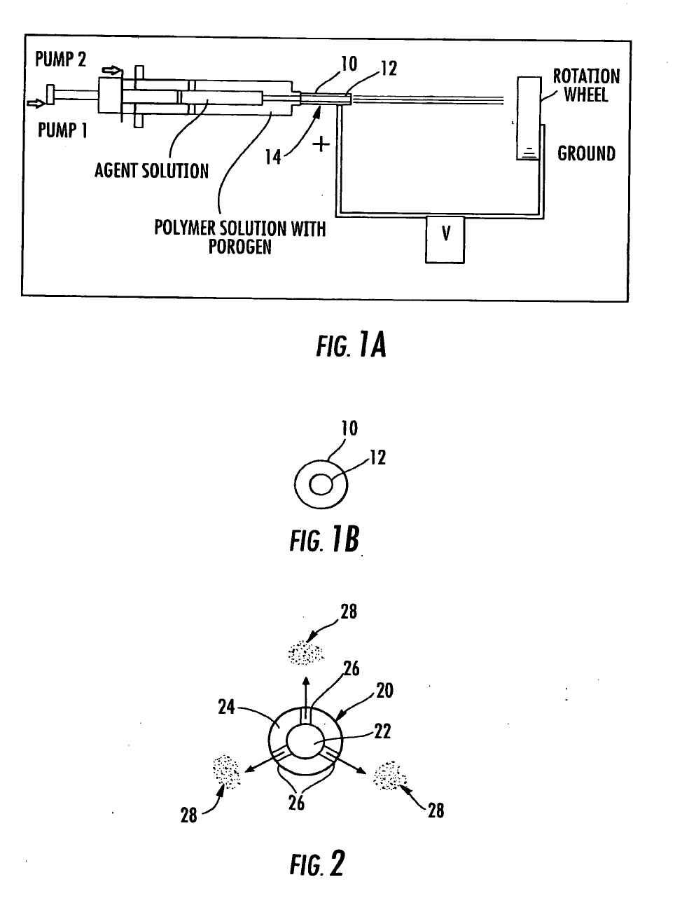

Referring to FIG. 1A, coaxial electrospinning by which embodiments of the present invention are produced, will be described. In the illustrated embodiment, two syringe needles 10, 12 are arranged concentrically at a location where a polymer jet is injected. In other words, a first needle 10 concentrically surrounds a second needle 12, as illustrated in FIG. 1B. A polymeric material is injected through the lumen of the first needle 10 and a material or agent that is to be encapsulated within a polymeric material fiber is injected through the lumen of the second needle 12. For example, a drug and/or other agents/materials, such as pharmacological materials, proteins, viruses, plasmid DNA, bacterial cells, and drug-loaded nanoparticles, etc., is dispersed in a solution (core phase) and is injected though the lumen of the second needle 12 so as to be encapsulated in the core of an electrospun fiber, the material of which is injected through the lumen of the first needle 10.

According to some embodiments of the present invention, a PCL solution, with PEG serving as porogen, serves as the shell phase of a fiber product and is injected through the lumen of the first needle 10. Another solution containing an agent is injected through the lumen of the second needle 12. As the two solutions are being injected through the respective needles 10, 12, a high electrical potential is applied at the needle tip 14. The polymer solution is extruded towards the ground, the solvent that dissolves the PCL dries in the air and the end product is a core-shell featured fiber.

As the process continues, a fibrous mesh will form. PEG, at MW around 1000-8000, does not form a true blend with PCL solution but is dispersed at different regions along the fibers. As the scaffold is introduced into a saline solution, the water soluble PEG is dissolved, leaving behind pores which serve as channels for the material of interest to leach out of (or elute from) the fiber.

Referring now to FIG. 2, a cross-section of a fiber 20, according to embodiments of the present invention, is illustrated. The illustrated fiber 20 includes a core 22 (which can be either a hydrophobic or hydrophilic core) inside of a fiber 24. The core 22 serves as a reservoir within the fiber 20. As the porogen leaches out of the fiber shell 24, channels 26 are created that extend between a surface of the fiber and the core 22. These channels 26 enable material within the core 22 to discharge from, and/or for material external to the fiber 24 to ingress into the core 22. In FIG. 2, an agent 28 is illustrated discharging from a fiber.

Embodiments of the present invention are not dependent on the diffusion ability of a material/agent within a fiber core through the polymer shell or on the degradation ability of the polymer. The material/agent release can be controlled by the concentration and the molecular weight of the porogen (PEG), which dictates the rate of pore formation.

Core-shell fibers, according to embodiments of the present invention have numerous advantages over conventional fibers. For example, a drug/protein of interest can be hydrophilic or hydrophobic, which allows the delivery of protein/growth factors and is not limited to hydrophobic drugs as is the case with conventional fibers. The addition of porogen allows an extra level of control over drug releasing rate rather than being dependent on diffusion/polymer degradation. Drugs in small quantities (such as seen with growth factor in micro and nanogram level) can be delivered via fibers according to embodiments of the present invention because the fibers do not rely on the partition of some proteins in the polymer phase for release to occur. In addition, viral/non-viral nanoparticles can be delivered only through core-shell fibers according to embodiments of the present invention and not through conventional nanofibers. Viral vectors are destroyed when dispersed in an organic solvent. Moreover, embodiments of the present invention can enable the combination of gene therapy with nanoscopic features offered by nanofibers.

According to some embodiments of the present invention, virus-encapsulated fibers produced via coaxial electrospinning are provided. These fibers can be formed into scaffolds that can achieve prolonged and localized gene delivery. The release of viral particles from the fibers can be finely controlled through the nanopores on the shell of the electrospun fibers. Moreover, sustained transgene expression can be achieved for at least one month, and mostly specific only to cells seeded on the scaffold. Applications for embodiments of the present invention include regenerative medicine. For example, fibrous materials according to embodiments of the present invention may produce a transfecting scaffold for infiltrating progenitor cells in vivo.

Virus-encapsulated fibers can be produced, in accordance with embodiments of the present invention, using the illustrated setup of FIG. 1A. A polymeric material is injected through the lumen of the first needle 10 and viral particles to be encapsulated within a polymeric material fiber are injected in a solution through the lumen of the second needle 12 so as to be encapsulated in the core of an electrospun fiber.

According to some embodiments of the present invention, fibers encapsulated with viable (i.e., live) bacterial cells via coaxial electrospinning are provided. According to embodiments of the present invention, bacterial cells can be successfully encapsulated into electrospun fibers without noticeable change in cell morphology and viability.

Fibers encapsulated with bacterial cells can be utilized in the development of biofilters. For example, biofilters in accordance with embodiments of the present invention can be utilized to remove pollutants from both water and airstreams. Fibers encapsulated with bacterial cells can be utilized in the development of long-term drug delivery implants.

Fibers encapsulated with bacterial cells can be produced, in accordance with embodiments of the present invention, using the illustrated setup of FIG. 1A. A polymeric material is injected through the lumen of the first needle 10 and bacterial cells to be encapsulated within a polymeric material fiber are injected in a solution through the lumen of the second needle 12 so as to be encapsulated in the core of an electrospun fiber.

### Example 1

Co-axially electrospun nanofibers were generated using a syringe-inside-syringe design. The needle gauges used for dispensing the polymer shell and protein core solutions were 30 G and 20 G, respectively. The flow rate was set at 1 mL/hr for the core solution and 3 mL/hr for the shell solution. The voltage gradient was adjusted from 10 to 15 kV, with the electrospinning distance fixed at 5 cm. Alignment of the core-shell PCL fibers was achieved by using a rotating drum (˜2,000 RPM) as the grounded target. Bovine serum albumin (BSA) was used for initial optimization. BSA solution (Sigma, USA) at 10 mg/mL in distilled water was used as the core solution and 7% w/v PCL (Mn 42,500, Sigma, USA) in 60:40 (v/v) dichloromethane: ethanol was used as the shell solution. The optimum flow rate ratio between the shell and core solutions was 3:1. Any large deviation from this ratio resulted in either a low protein loading efficiency or electrospraying. Different formulations of PCL and PEG (Mn of 950-1050, Mn 3400 and Mn 8000 and PEG concentrations of 1 mg/mL and 20 mg/mL) were studied. The electrospinning process was continued until 1 mg of BSA was loaded into each sample. The encapsulation of recombinant human platelet derived growth factor-bb (Cordis Corporation) was performed using the same technique, and each scaffold was loaded with 20 μg of the growth factor. The experiments used poly(caprolactone) (Mn. 42,500) solution (7% wt. in 70:30 volume ratio of chloroform:ethanol). The flow rate of the outer shell solution is 4 ml/hr.

For the protein release kinetics study, fibrous scaffolds (n=3) were each incubated in 2 mL of PBS solution at 37° C. The supernatant was then removed and replenished with fresh PBS solution at predetermined time intervals. The amount of BSA present in the supernatant was determined by microBCA protein assay, while the amount of PDGF-bb released was analyzed by ELISA.

The bioactivity of the released PDGF-bb was measured by the proliferation of NIH 3T3 fibroblast. The concentration of PDGF-bb released from the PCL fibers (n=3) was first determined using ELISA, and then diluted to 10 ng/mL for addition into the cell cultures. The proliferation of 3T3 fibroblasts incubated with the addition of supernatant samples was compared with those cultured in complete medium with 10 ng/mL of fresh PDGF-bb (positive control), as well as those in complete medium only (negative control).

The FITC-RSA loaded PCL fibers were used to study fiber alignment and the quality of protein encapsulation. The size and surface morphology of the PCL fibers were analyzed using SEM, and the core-shell structure was verified by TEM.

The control of drug release is designed around PEG's function as a porogen in the shell of the protein loaded nanofibers. Low molecular weight PEGs have been shown to be non-cytotoxic, filterable by kidneys and are able to function as a porogen, creating pores in the scale of 500 nm. By incorporating PEG into the shell of PCL nanofibers to induce pore formation and fiber swelling, the release of the encapsulated proteins can be controlled in a manner that is independent of loading concentration and core diameter.

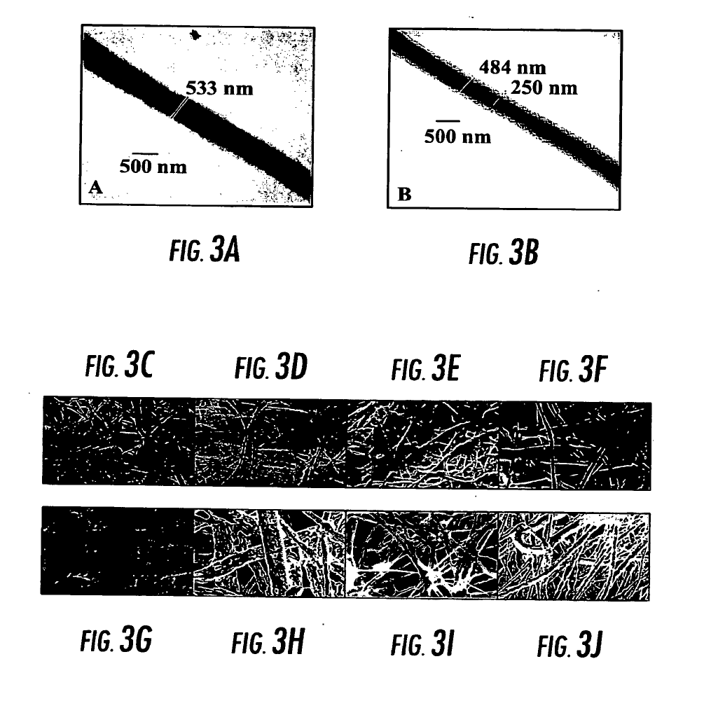

FIGS. 3A-3J are electron microscopy images of electrospun nanofibers according to some embodiments of the present invention. FIG. 3B reveals the presence of the core-shell feature in the nanofibers through the encapsulation of 1% w/v uranyl acetate. The average diameter of the nanofibers is approximately 500 nm, with an average core diameter of 250 nm. FIG. 3A illustrates a core-shell nanofiber without uranyl acetate.

The surface morphologies and fiber diameters of nanofibers produced with different PCL/PEG blends (MW 1000, MW 3400 and MW 8000) were monitored over a period of 30 days. In general, the incorporation of PEG increased the degree of fiber swelling in all three blends and produced noticeable pore formation in the PCL/PEG 3400 and 8000 blends (FIGS. 3H and 3J). Incorporation of FITC-PEG (MW 3000) suggested a complete leaching of PEG by day 3 (data not shown). No significant changes in surface morphology or fiber diameter were seen in PCL fibers without any PEG. The most significant amount of fiber swelling and pore formation occurred in PCL/PEG 3400 blend (FIG. 3H). This suggests that PEG 3400 is more efficient as a porogen than PEG 1000 and PEG 8000 blends. The significance of controlling the rate of pore formation is demonstrated by the study of BSA release from different blends of PCL/PEG fibers.

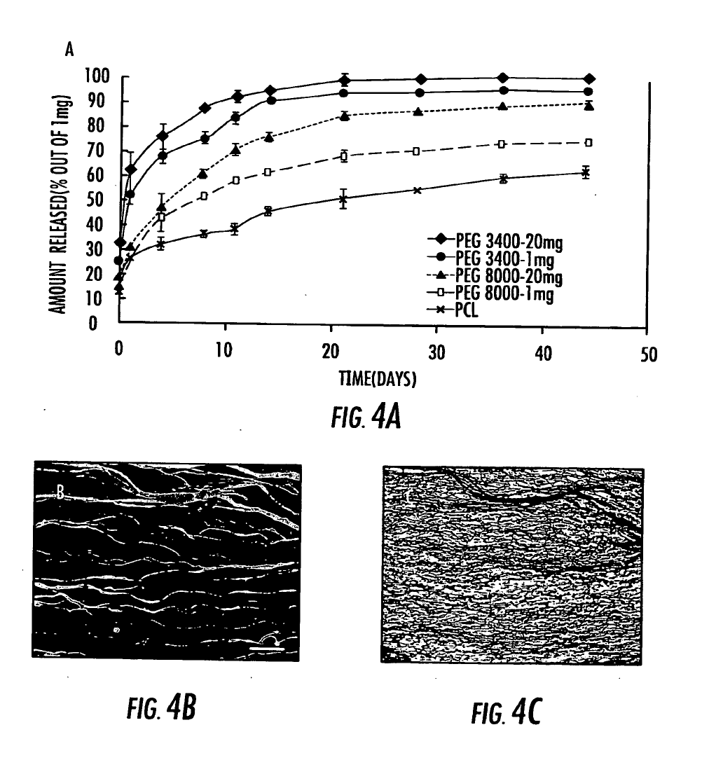

FIG. 4A is a graph that illustrates the controlled release of encapsulated bovine serum albumin from PCL and various formulations of PEG blended PCL nanofibers, according to some embodiments of the present invention. FIG. 4B is a fluorescent microscopy image of aligned FITC-BSA loaded PCL nanofibers. FIG. 4C is a corresponding phase image of aligned FITC-BSA loaded PCL nanofibers. The incorporation of PEG into PCL nanofibers increased the BSA release rate, in a concentration and molecular weight dependent fashion (FIG. 4A). PEG 3400, more effective as a porogen than PEG 8000, also induced a faster BSA release rate (FIG. 4A). In addition, increasing the PEG concentration from 1 to 20 mg/mL also resulted in faster release kinetics. Correlation of PEG induced pore formation and BSA release rate suggested that drug release takes place in a pore dependent fashion.

The quality of the controlled release nanofibers was examined through the encapsulation of FITC-BSA. The fluorescent images revealed a uniform distribution of BSA, with no sign of aggregation and discontinuity of FITC-BSA within the core of the fibers (FIGS. 4B and 4C). The encapsulation efficiency (100%) and the loading level (5% of the fiber weight) in this study showed that co-axial electrospinning can significantly improve the protein loading ability of electrospun fibers. Fluorescent images also demonstrated that fiber alignment is possible. Aligned nanofibers have been shown to induce cell alignment on the surface of the nanofibers and even enhance the extracellular-matrix production in fibroblasts. Achieving alignment in protein loaded nanofibers can therefore influence cellular orientation through nano-topographical cues and enhance cell differentiation and matrix production through the delivery of bioactive agents leveling a local and sustained manner.

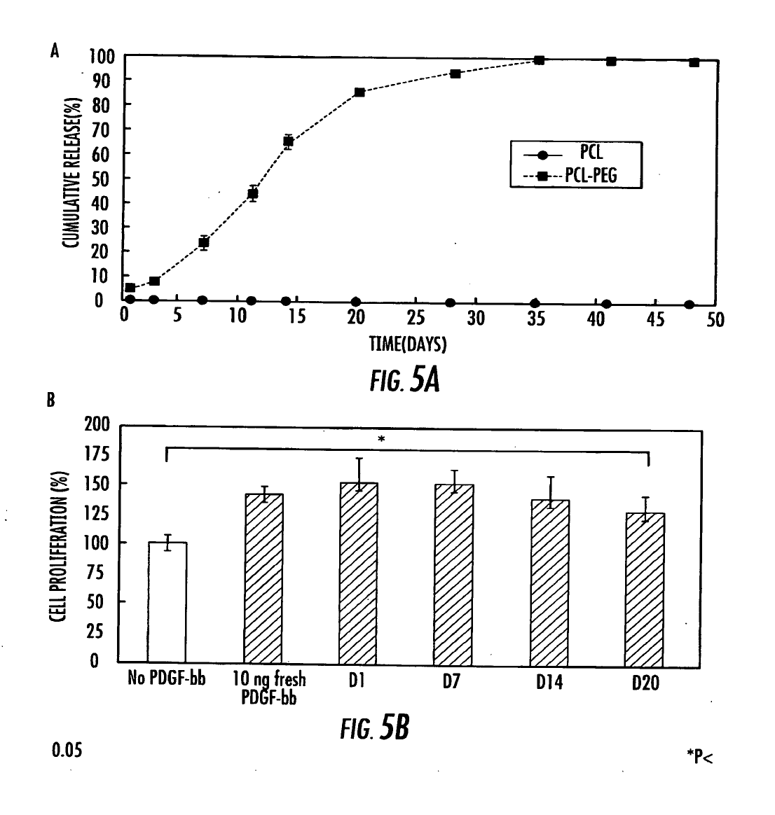

FIG. 5A is a graph that illustrates the controlled release of encapsulated PDGF-bb from PCL nanofibers and PCL-PEG (7% PCL+20 mg/mL PEG MW 3400)) nanofibers. FIG. 5B is a graph that illustrates bioactivity of PDGF-bb released into the supernatant solution based on the enhanced proliferation rate of NIH 3T3 cells. Another significant finding is the preservation of the bioactivity of the encapsulated growth factor. PDGF-bb was loaded into PCL and PCL/PEG (20 mg/mL of MW 3400 PEG) nanofibers at 40% efficiency. The encapsulated growth factor reached 100% release in 35 days with a relatively linear release profile (FIG. 5A) from the PCL/PEG fibers. In contrast, only a very small amount of growth factor (<1%) was released from the same PCL core-shell nanofibers without PEG in the shell. This suggests that the encapsulated PDGF-bb, unlike BSA, could not function as a porogen, and that the release must rely on pore formation on the fiber surface. The bioassay based on the proliferation rate of NIH 3T3 cells also revealed the preservation of PDGF-bb bioactivity (FIG. 5B). With its ability to achieve high protein loading level and controlled release of bioactive proteins from nanofibers, co-axial electrospinning represents a powerful technique to advance the field of tissue engineering and regenerative medicine.

### Example 2

Adenoviruses are double-stranded DNA viruses. They have icosahedral capsids with twelve vertices and seven surface proteins. The virion is non-enveloped, spherical and about seventy to ninety nm in size. The adenovirus construct (an adenovirus encoding GFP plasmid from Vector Biolab) used in this example has a CMV promoter and transfects cells to produce green fluorescent protein. When cells are producing green fluorescent protein, they will fluoresce green under the fluorescent microscope.

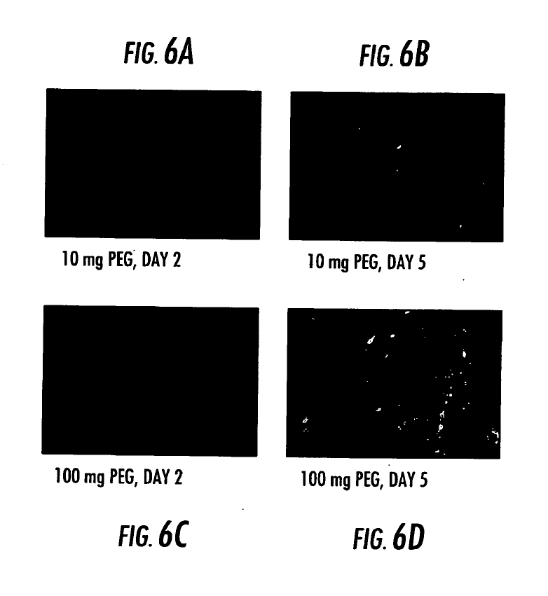

The adenovirus (1×10̂6 IFU/PFU) is encapsulated into the core of the core-shell nanofibrous scaffold. Two different polymer compositions have been utilized in which different amounts of MW3400 PEG is blended with the 10% PCL solution (10 mg/ml PEG and 100 mg/ml PEG) in 75/25 volume ratio of chloroform/ethanol. At day 1, 1×10̂5 of bovine pulmonary artery smooth muscle cell is seeded onto each nanofibrous scaffold. At day 2 and 5, fluorescent images were taken on the cells seeded on the nanofibrous scaffold (FIGS. 6A-6D). No transfection was noticeable at day 2 in both polymer compositions. At day 5, the smooth muscle cells that are seeded onto the viral vector encapsulated nanofibrous scaffold started to produce the green fluorescent protein. This provided evidence that the viral vectors encapsulated in the core shell nanofibers are capable of leaching out of the fibers and transfecting the cells that are seeded onto the scaffold. This is the first work that suggests that viral vectors can be encapsulated into nanofibrous scaffold and maintains its function in gene therapy.

### Example 3

Poly (ε-caprolactone) (Mw-65,000, Sigma, USA) was dissolved in 75:25 (v/v) ratio of chloroform:ethanol at 10% wt. and was used as the PCL polymer solution. Poly (ethylene glycol) (PEG, Mw 3,400, Union Carbide Corporation, USA) was the porogen. Two types of adenovirus (type V, E1/E3 deleted, encoding for green and red fluorescent protein) were purchased from Vectorbiolabs, USA. Virus purification and quantitation kit (ViraBind™ Adenovirus Purification Kit and QuickTiter™ Adenovirus Quantitation Kit from Cellbiolabs, USA) were used to purify and quantify virus titer. Goat anti-adenovirus fluorescein isothiocyanate conjugate (Fitzgerald Industries Internationals Inc, USA) was used at a dilution of 1:100 to label the encapsulated adenovirus. Uranyl acetate (Electron Microscopy Science) was dissolved in distilled water at 1% wt. to serve as a contrast agent in transmission electron microscopy. Minimum essential medium (MEM with Earl's salt and glutamine, Gibco, USA) supplemented with 10% fetal bovine serum (Mediatech, USA) was used as cell culture medium and viral titer diluent. HEK 293 cell proliferation was determined by using cell proliferation WST-1 reagent (Roche Molecular Biochemicals, USA). Collagenase type 1 (Sigma, USA) was used to enhance cell trypsinization from the scaffolds. Phosphate buffered saline solution (PBS, pH 7.4, Gibco, USA) was used to incubate samples for surface morphology studies. 4′,6-diamidino-2-phenylindole, dihydrochloride (DAPI, Invitrogen) was used to label cell nucleus in the localized transduction studies.

Virus in MEM solution with 0.1% bovine serum albumin and PCL solution in organic solvent were dispensed through two co-axially arranged needles and exposed to high voltage gradient (15 kV) between the needles and a designated ground. As the solutions were dispensed (MEM solution is in the inner needle while the PCL solution is in the outer needle), the high voltage gradient drew the solutions into microscale fibers before the solution reaches the designated ground. Several setup parameters were important to ensure efficient virus encapsulation: the distance to ground was kept at 10 cm, the needle gauges for core and shell solutions were 30 G and 18 G, and the solution flow rates were 1 mL/hr (core) and 6 mL/hr (shell). 100 μL of adenovirus (10⁷ IFU/PFU/ml) solution was encapsulated into a 1.5 cm×10 cm strip. The samples were then cut into 1.5 cm×1 cm dimension (10⁷ IFU/PFU/sample) and sterilized overnight in PBS solution with 25 μg/ml of fungizone and 10 U/ml of penicillin/streptomycin. Each scaffold was 100 μm thick and weighed approximately 5 mg. Fibrous scaffolds with different PEG concentration (0, 0.07, 0.7 and 7%) were prepared with the same procedure.

In order to verify that the adenovirus had been efficiently encapsulated and uniformly distributed in the fibers, the viral vectors (10⁷ IFU/PFU/mL) were conjugated with anti-adenovirus-FITC (1:100 dilution) prior to encapsulation. The conjugated viral vectors were then filtered through a 0.45 μm filter, washed with PBS and eluted with 25 mM Tris buffer. The conjugated adenovirus was then encapsulated as described above and imaged under fluorescence microscope.

The two most important physical characteristics of the co-axial fibrous scaffold in this study were the core-shell and nano-porous surface features. These fiber characteristics were examined by using transmission and scanning electron microscopy (Hitachi HF-2000 and FEI XL30 SEM-FEG). In order to illustrate the presence of core in the fibers under TEM, 1% wt. uranyl acetate was encapsulated in the same conditions as previously reported. The electrospun fibers were then mounted onto TEM grids directly and imaged. The surface morphology changes on the fibrous scaffold were studied as a function of different porogen concentrations (0, 0.07, 0.7 and 7.0%). The scaffolds were first incubated in PBS solution at 37° C., dried under vacuum overnight and coated with 4 nm of gold (Bal-Tec MED 020) prior to imaging under SEM. These changes in physical features govern the fibers' ability to encapsulate and release the viral vectors.

HEK 293 cells (passage 17-25) were cultured in complete minimum essential medium and used as the model cell type for viral gene delivery. There are several aspects of cell infection that this example focuses on: cumulative release of adenovirus into the supernatant over time, cell infection through controlled release of adenovirus and cell seeding onto the scaffolds, and cell proliferation rate when cultured on the virus releasing scaffolds. Cell transgene expression was studied as a function of PEG (0, 0.07, 0.7 and 7%) formulation. Virus encapsulated scaffolds (10⁵ IFU/PFU/sample, n=3) were incubated in 1 mL of complete medium at 37° C. and 5% CO₂. Controlled release of the adenovirus was performed by removing and replenishing the supernatant at predetermined time points (Day 7, 14, 21, 28 and 35). The viral titer in the supernatant solutions was determined by performing end point dilution assay.

Cell infection through controlled release of adenovirus was done by exposing 5×10⁵ 293 cells (n=3) cultured on tissue culture grade polystyrene (Corning) to scaffold supernatant overnight. The viral supernatants were replaced with regular 293 medium and the cells were cultured for 7 days. Cell transduction rate was determined by flow cytometry studies (BD FACScan™ Flow Cytometer, BD Biosciences). Data were acquired and analyzed using CellQuest Pro software (BD Biosciences).

To evaluate how the virus encapsulated scaffold transduce seeded cells overtime, virus encapsulated scaffolds (n=3 for every time point) were incubated in medium and removed for cell seeding at predetermined time points (Day 1, 7, 14, 21 and 28). Cell transduction via cell seeding was performed by suspending 5×10⁵ 293 cells in 100 μL of medium and pipetted onto the virus encapsulated scaffolds. The cells were allowed to attach onto the scaffolds for 1 hour in 37° C. and 5% CO₂. The scaffolds were then transferred into new wells and cultured for 7 days. The cell seeding efficiency was approximately 80% in all conditions. To efficiently remove the seeded cells from the scaffolds for flow cytometry study, the scaffolds were incubated in 500 μg/mL of collagenase type 1 in PBS solution for 1 hour and subsequently trypsinized. Approximately 90% of the seeded cells were removed. The cell infection rate of the seeded cells was measured by flow cytometry.

Cell proliferation rate on the virus encapsulated scaffolds were studied by culturing 5×10⁵ 293 cells on scaffolds (n=3) produced with different formulations (0, 0.07%, 0.7%, and 7% PEG). A set of blank PCL scaffolds was used as positive control to the virus encapsulated scaffolds. WST-1 proliferation assay was performed every 2 days. WST-1 assay was performed according to the protocol provided (Roche Molecular Biochemicals) with cells being exposed to the reagents for 2 hours. The absorbance levels of the supernatants were measured with a microplate reader (Fluostar optima, BMG labtech) at 450 nm.

The ability of the virus encapsulated scaffolds in localizing cell infection was investigated in-vitro through three co-culture studies. In the first setup, scaffolds (0.7% PEG formulation) encapsulated with 10⁵ IFU/PFU/sample was placed in a 3 μm transwell with a monolayer of 5×10⁵ cells cultured in the bottom of the well. At day 5 the cells were trypsinized, resuspended in PBS and analyzed with flow cytometry. In second study, virus encapsulated scaffolds were first seeded with 5×10⁵ cells, then transferred to a 3 μm transwell and co-cultured with a monolayer of 5×10⁵ cells. The cells cultured on the scaffold and the cells cultured in the monolayer were trypsinized on day 5 and analyzed with flow cytometry. The third study consisted of co-culturing two types of scaffolds (GFP-CMV-AV and RFP-CMV-AV) separately with a transwell. 5×10⁵ cells were seeded on each of the scaffolds and the co-culture was maintained for 5 days. On day 5, the scaffolds were fixed in 4% paraformaldehyde, stained for cell nucleiwith DAPI counterstain and imaged under fluorescence microscope.

The virus encapsulation process of co-axially electrospun fibers produced in different conditions is evaluated qualitatively in Table 1.

TABLE 1

Shell flow rate

| Core flow rate | 10 mL | 8 mL | 6 mL | 4 mL |
|---|---|---|---|---|
| 6 mL | X | X | X | X |
| 4 mL | Z | X | X | X |
| 2 mL | Y | Z | Z | X |
| 1 mL | W | W | Y | Z |
| 0.5 mL | W | W | W | W |

X Ratio < 2:1 Cannot form fibers
Z Ratio = 3:1 Phase separation during electrospinning
W Ratio > 8:1 Low encapsulation efficiency
Y Ratio between 5:1 to 6:1 Ideal encapsulation condition

The success of virus encapsulation was evaluated by inspecting how the solutions were sheared into microfibers and examining whether the FITC-labeled adenovirus particles are detectable in the fiber product. The flow rate ratio between the two phases is a major deciding factor on the virus encapsulation efficiency. At a flow rate under 2:1 shell to core ratio, no electrospun fibers can be produced and an aggregate formed at the needle tip. When the flow rate ratio was increased to 3:1, FITC-labeled adenovirus particles were encapsulated into the electrospun fibers, but there was noticeable phase separation in the polymer solutions, an indication that the viral particle encapsulation process was less than optimal. As the flow rate ratio was increased to between 5:1 and 6:1, there was no noticeable phase separation and the FITC-labeled viral particles were detectable through fluorescence microscope. Further increase in flow rate ratio (ratio greater than 8:1) reduced the virus encapsulation concentration and therefore fluorescent intensity.

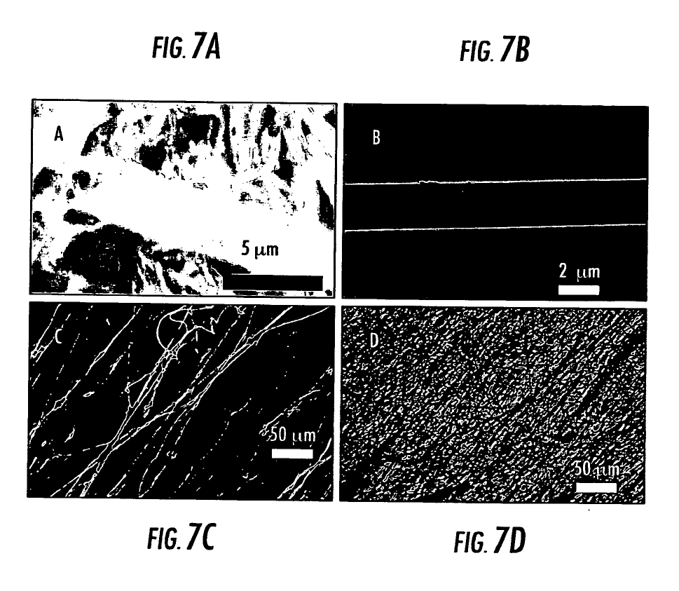

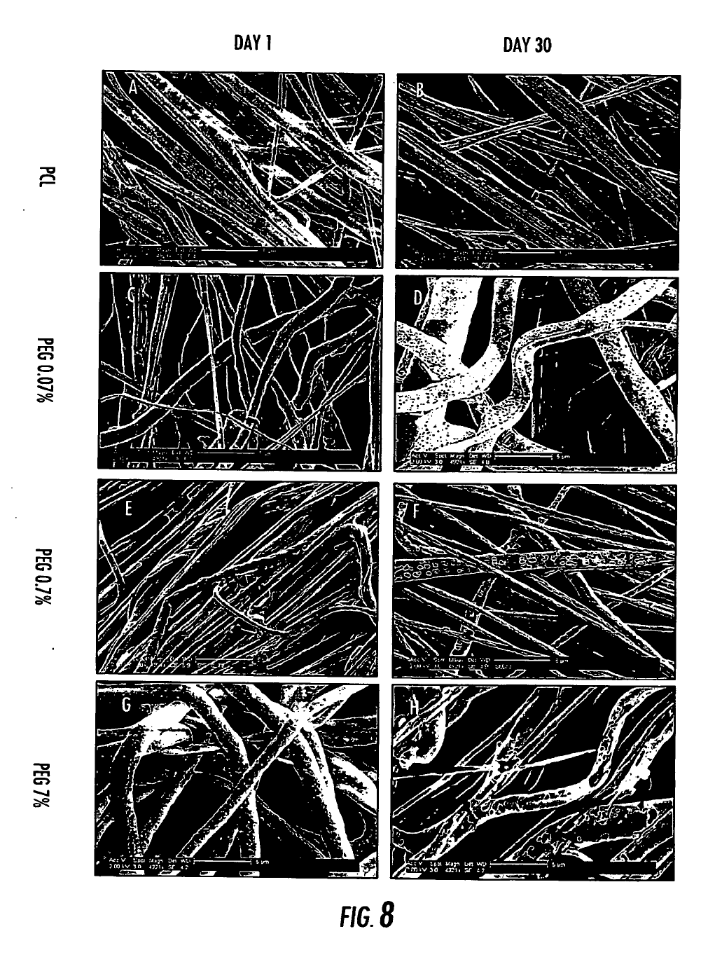

The fiber core-shell features and the adenovirus virus distribution amongst the electrospun fibers are reported in FIGS. 7A-7D. Freeze dried-fractured fibers and uranyl acetate encapsulated fibers showed that the average overall diameter of the fiber ranges between 2-3 μm, with the core diameter remains around 1 μm (FIGS. 1A and 1B). Porogen concentration has insignificant effects on the diameter of the fibers. As shown in FIGS. 1C and 1D, FITC-labeled adenovirus was uniformly and efficiently encapsulated throughout the fibers. Fibers encapsulating none-labeled virus did not display detectable autofluorescence.

To enhance the release of encapsulated viral vectors, this design takes advantage of the fact that small molecular weight poly (ethylene glycol) can be uniformly dispersed into the polymeric solution, and capable of leach out to create porous structures on the fiber surfaces. FIGS. 8A-8H illustrate the influence of porogen on the surface morphology changes to the virus encapsulated fibers. Previous work has shown that poly(ethyl glycol) at Mw 3,400 is rapidly released (100% in 5 days) from the shell of the electrospun fibers and is capable of creating pores on the scale of a few hundred nanometers. FIGS. 8A-8H report the influence of different concentration of PEG on surface morphology. As expected, fibers without porogen showed very little swelling and surface morphology changes (FIGS. 8A and 8B). Fibers produced with the formulation of 0.07 and 0.7% PEG exhibited a very significant level of pore formation on the surface of the fiber by day 30 (FIGS. 8D and 8F) as opposed to no pore formation on day 1 (FIGS. 8C and 8E). High magnification of the fiber surface revealed that the pore size was approximately 200 μm. At the highest PEG concentration (7% wt.), SEM images of the fiber surface suggested a significant level of fiber degradation (arrow) in additional to pore formation (FIG. 8H). In short, the degree of pore formation and changes in fiber surface morphology has a direct correlation with the concentration of porogen incorporated into the fibers.

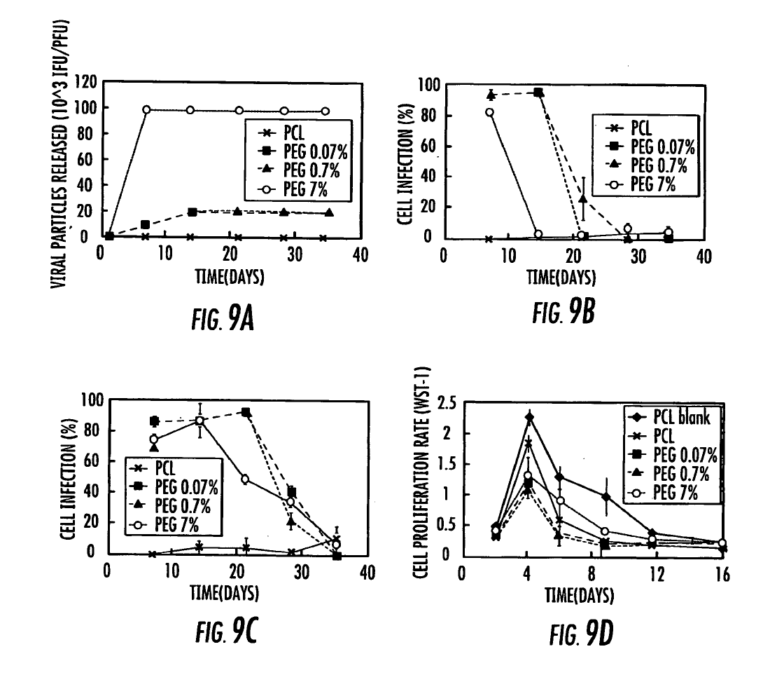

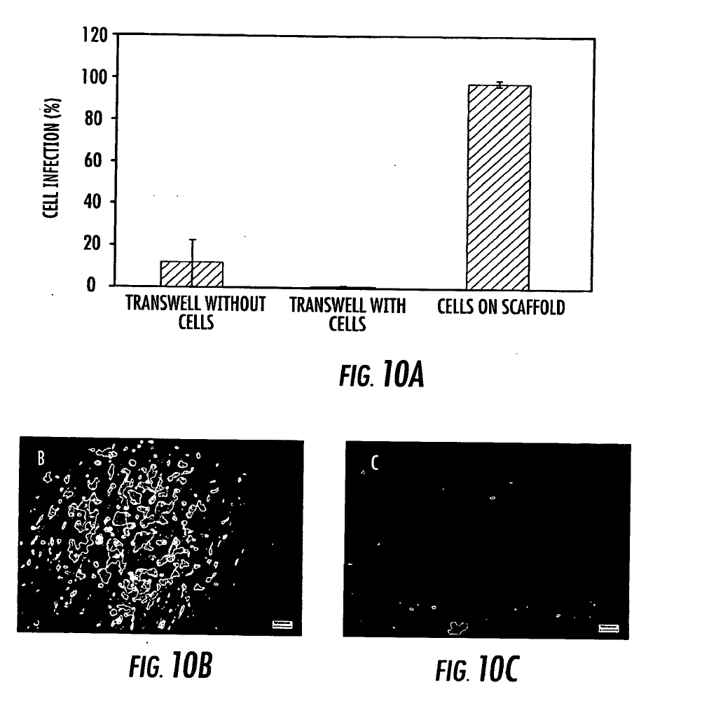

In order to evaluate how well the virus-encapsulated electrospun fibers serve as a transducing tissue engineering scaffold, the focus was on four parameters: controlled release of adenovirus, cell transduction from scaffold supernatant, cell transduction when seeded on the scaffold and cell proliferation rate when cultured onto the scaffold. Different shell formulations (0, 0.07, 0.7 and 7% wt. of PEG) were also studied to correlate pore formation to cell transduction ability. End point dilution assay based on the scaffold supernatant suggests that the controlled release of the adenovirus does follow a PEG concentration dependent trend. Close to 100% of the adenovirus was released from the 7% PEG samples while the total amount release in the 0.07 and 0.7% remained to be around 20% (FIG. 9A). Porogen-less scaffold did not have significant level of virus released into the supernatant (FIG. 9A). Cell transduction by overnight exposure to scaffold supernatant solution suggests consistent results with the end point dilution assay. Close to 90% transduction is seen from PEG incorporated scaffolds in the first two weeks followed by a drastic drop to 0% in subsequent weeks (FIG. 9B). Transduction was only seen in the first week in the 7% wt. PEG sample, a finding that implies that complete exhaustion of encapsulated viral particles has occurred (FIG. 9B). However, when cells were seeded onto the virus-encapsulated scaffold (at a density of 5×10⁵/sample, cultured for 1 week), the seeded cells expressed transgene expression for over 1 month (FIG. 9C). Despite the end point dilution data suggesting that close to 100% of the encapsulated virus had been released, the 7% wt. PEG sample was still able to transduce cells at a significant level (FIG. 9C). The PEG-less sample exhibits a low level of cell transduction throughout the culture period (FIG. 9C).

The data presented in FIGS. 8A-8H and FIGS. 9A-9D suggest that the created pores are crucial to achieving transgene expression, and that the encapsulated viral particles can leach out through the pores and transduce cells. Viral particles encapsulated into the porogen-less fibers remained trapped inside, and both the end point dilution and cell transduction data suggest that there are no viral particles released. Fibers with 0.07 and 0.7% wt. PEG experienced intermediate levels of pore formation, enabling the fibers to release approximately 20% of the encapsulated virus over 2 weeks. The trapped viral vectors in both of samples were still capable of achieving transgene expression over one month in the cells seeded on the scaffolds. Fibers with high PEG concentration (7% wt.) experienced more drastic changes in their surface morphology and have released most of the viral particles in the first two weeks. Interestingly, the remaining viral particles in the fibers still induced transgene expression close to one month. This discrepancy in value can possibly be attributed to the lack of sensitivity in end point dilution assay.

To determine whether the encapsulated virus will prohibit growth in the seeded cells, the cell proliferation rate of the cells seeded on various PEG formulations was evaluated over 2 weeks. WST-1 proliferation rate of the cultured cells suggested a general trend of an initial fast increase followed by a quick drop in metabolic activity in all samples, including a set of blank, non-virus encapsulated PCL scaffolds (FIG. 9D). The comparison between various groups suggest there is a certain level of cell transduction related decrease in metabolic activity, though the difference between PEG incorporated and PEG-less samples was small (FIG. 9D). WST-1 proliferation assay suggested that despite a certain level of transduction related decrease in cell metabolic activity, the cells seeded on the virus encapsulated scaffold were still capable of proliferation and populated the fibrous scaffold.

The ability of the electrospun fibers to localize the cell transduction to its close proximity was evaluated through a set of co-culture experiments. When a virus loaded scaffold (0.7% wt. PEG) was cultured with a monolayer of cells (separated by a 3 μm transwell) for 5 days, approximately 10% of the cells exhibited transgene expression (FIG. 10A). In comparison, when cells were first seeded onto the scaffold, the co-culture monolayer exhibited close to 0% transgene expression, while 97% of the seeded cells were transduced (FIG. 1A). Cell transduction seems to prefer cells in closer proximity than farther, as suggested by the drastic difference in transgene expression in the cells seeded on the scaffold (97% transduced) versus the monolayer culture (0.15% transduced). This finding is conceivable because cells seeded on the fibers are spreading across the pores, making it possible to achieve transduction in the cells on the scaffold but not the monolayer culture. This theory was further put to test by seeding cells onto two scaffolds encapsulated with different viral vectors (Ad-CMV-GFP and Ad-CMV-RFP) and co-cultured for 5 days. Fluorescence microscopy images shown in FIGS. 10B and 10C reveal that the cell transduction was specific (cells seeded on the two scaffolds exhibited different reporter genes) and localized (very little cross transduction). This finding therefore suggests that cell transduction from virus-encapsulating scaffold is a close proximity phenomenon.

Co-axial electrospinning has been shown in this example to be an innovative method to create a tissue engineering scaffold capable of prolonged cell transduction. The attractiveness in this design is that adenovirus is exposed to the cells only when pores are formed on the fiber surface, as opposed to simply dispersing the viral vectors throughout the scaffold. The co-axial electrospinning design gives greater control over cell transduction and is possibly more capable of reducing virus dissemination and immune response.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of this invention as defined in the claims. The invention is defined by the following claims, with equivalents of the claims to be included therein.

1. A layer of fibrous material, comprising:
a plurality of fibers, wherein each fiber comprises a core and a shell surrounding the core, wherein the shell includes a plurality of channels that extend from an outer shell surface to the core; and
an agent encapsulated within the core, wherein the agent discharges from the core through the channels at a controlled rate.

2. The layer of fibrous material of claim 1, wherein the shell surrounding the core of each fiber comprises a polymer.

3. The layer of fibrous material of claim 1, wherein the shell surrounding the core of each fiber comprises poly(caprolactone).

4. The layer of fibrous material of claim 1, wherein the agent is selected from the group consisting of pharmacological materials, proteins, viruses, plasmid DNA, bacterial cells, and drug-loaded nanoparticles.

5. The layer of fibrous material of claim 1, wherein the channels are formed by porogen material disposed within the shell.

6. The layer of fibrous material of claim 5, wherein the porogen material comprises polyethylene glycol (PEG).

7. The layer of fibrous material of claim 1, wherein the plurality of fibers are aligned.

8. (canceled)

9. The layer of fibrous material of claim 1, wherein the fibers are selected from the group that includes nanofibers and microfibers.

10. (canceled)

11. A tissue engineering scaffold, comprising:
a plurality of fibers, wherein each fiber comprises a core and a shell surrounding the core, wherein the shell includes a plurality of channels that extend from an outer shell surface to the core; and
viral particles encapsulated within the core, wherein the viral particles discharge from the core through the channels at a controlled rate.

12. The tissue engineering scaffold of claim 11, wherein the viral particles are substantially uniformly distributed within the core.

13. The tissue engineering scaffold of claim 11, wherein the shell surrounding the core of each fiber comprises a polymer.

14. The tissue engineering scaffold of claim 11, wherein the shell surrounding the core of each fiber comprises poly(caprolactone).

15. The tissue engineering scaffold of claim 11, wherein the channels are formed by porogen material disposed within the shell.

16. The tissue engineering scaffold of claim 15, wherein the porogen material comprises polyethylene glycol (PEG).

17. The tissue engineering scaffold of claim 11, wherein cells seeded on the scaffold exhibit transgene expression for a predetermined period of time.

18. The tissue engineering scaffold of claim 11, wherein the fibers are selected from the group that includes nanofibers and microfibers.

19. (canceled)

20. The tissue engineering scaffold of claim 11, wherein the fibers are aligned.

21. (canceled)

22. A layer of fibrous material, comprising:
a plurality of fibers, wherein each fiber comprises a core and a shell surrounding the core, wherein the shell includes a plurality of channels that extend from an outer shell surface to the core; and
viable bacterial cells encapsulated within the core.

23. The layer of fibrous material of claim 22, wherein the bacterial cells secrete material through the one or more channels at a controlled rate.

24. The layer of fibrous material of claim 22, wherein the bacterial cells absorb material external to the fibers through the one or more channels.

25. The layer of fibrous material of claim 22, wherein the bacterial cells discharge from the core through the one or more channels at a controlled rate.

26. The layer of fibrous material of claim 22, wherein the bacterial cells are encapsulated within the core in an aqueous solution.

27. The layer of fibrous material of claim 22, wherein the shell surrounding the core of each fiber comprises a polymer.

28. The layer of fibrous material of claim 22, wherein the shell surrounding the core of each fiber comprises poly(caprolactone).

29. The layer of fibrous material of claim 22, wherein the channels are formed by porogen material disposed within the shell.

30. The layer of fibrous material of claim 29, wherein the porogen material comprises polyethylene glycol (PEG).

31. The layer of fibrous material of claim 22, wherein the fibers are microfibers.

32. The layer of fibrous material of claim 22, wherein the fibers are aligned.

33. (canceled)

34. A method of forming a fibrous material, comprising co-axially electrospinning first and second solutions to form a plurality of fibers, wherein the first solution forms a fiber core and the second solution forms a shell surrounding the core, wherein the first solution includes an agent selected from the group consisting of pharmacological materials, proteins, viruses, plasmid DNA, bacterial cells, and drug-loaded nanoparticles, and wherein the second solution is a polymeric solution that includes porogen material, wherein the porogen material is configured to leach from the shell and form one or more channels that extend from an outer shell surface to the core.

35. The method of claim 34, wherein the second solution includes poly(caprolactone).

36. The method of claim 34, wherein the porogen material is polyethylene glycol (PEG).

37. The method of claim 34, wherein the plurality of fibers are aligned.

38. (canceled)

39. The method of claim 34, wherein the fibers are selected from the group that includes nanofibers and microfibers.

40. (canceled)
